(19) United States
(12) Patent Application Publication
(10) Pub. No.: US 2021/0128338 A1
(43) Pub. Date: May 6, 2021

(54) JOINT STABILIZATION GARMENTS

(71) Applicant: BETTER WALK, INC., Atlanta, GA (US)

(72) Inventors: Partha Sarathy UNNAVA, Atlanta, GA (US); Tyler Jack Prescott HARMON, Decatur, GA (US)

(21) Appl. No.: 16/641,509

(22) PCT Filed: Aug. 22, 2018
(86) PCT No.: PCT/US18/47619

§ 371 Date: Feb. 24, 2020

Related U.S. Application Data
(63) Continuation-in-part of application No. 29/631,819, filed on Jan. 3, 2018, now Pat. No. D883651.
(60) Provisional application No. 62/548,609, filed on Aug. 22, 2017.

(51) Int. Cl. A61F 5/01; A41B 11/00; A41D 27/10; A41D 13/06; A41D 13/08

(52) U.S. Cl. CPC A61F 5/0127; A41B 11/003; A41D 27/10; A41D 2400/32; A41D 13/08; A61F 5/013; A61F 5/0123; A41D 13/065

(57) ABSTRACT

Technical garments including joint stabilization socks, sleeves, and other garments are disclosed. The exemplary garments disclosed herein include regions with material properties, such as tensile strengths, that are different from other regions of the garment. Particular regions of the exemplary garments include interwoven materials with substantially high tensile strengths for providing resistance/compression to targeted body parts (e.g., ankles, knees, shoulders, etc.) for preventing undesirable movement (e.g., that mimics medically-relevant taping patterns). In particular embodiments, the regions with higher tensile strengths are interwoven or integrated into a base material, such that the garment can be comfortably worn while also providing compression and support to targeted body parts.

### CROSS REFERENCE TO RELATED APPLICATIONS

This application claims the benefit of, and priority to, International Patent Application No. PCT/US18/47619, filed on Aug. 22, 2018, and entitled “Joint Stabilization Garments,” which claims the benefit of, and priority to, U.S. Provisional Patent Application No. 62/548,609, filed Aug. 22, 2017, and entitled “Joint Stabilization Sock,” and is a continuation-in-part of and claims priority to U.S. Design patent application Ser. No. 29/631,819, filed Jan. 3, 2018, and entitled “Joint Stabilization Sock,” the disclosures of which are incorporated by reference in their entireties as if the same were fully set forth herein.

This application is related to and incorporates herein by reference the following design patent applications:

U.S. Design patent application Ser. No. 29/660,787, filed Aug. 22, 2018 and entitled JOINT STABILIZATION KNEE SLEEVE; and

U.S. Design patent application Ser. No. 29/660,783, filed Aug. 22, 2018 and entitled JOINT STABILIZATION ELBOW SLEEVE.

### TECHNICAL FIELD

The present disclosure relates to technical garments, and more particularly to joint stabilization garments.

### BACKGROUND

Traditional practices for injury prevention in joints, muscles, and bones include athletic taping. Athletic taping is a conventional process where portions of the body (e.g., joints, muscles, bones, etc.) are maintained or supported in positions by applying tape directly to the surrounding skin. When applied in particular patterns, athletic tape may provide compression to the targeted body regions; however, athletic tape is difficult to apply properly without professional assistance, loses adherence and form (thus losing the pattern's initial compressive benefits), may irritate skin, and is generally wasteful as tape cannot be reused.

Therefore, there is a long-felt but unresolved need for an alternative to athletic taping that not only provides similar compression benefits to taping but also overcomes its many shortcomings.

### BRIEF SUMMARY OF DISCLOSURE

Briefly described, and according to one embodiment, aspects of the present disclosure generally relate to technical garments.

The present disclosure describes embodiments of joint stabilization garments manufactured from materials with variable material properties. In one embodiment, the garments may include a plurality of portions or regions, where each portion or region includes a particular material with particular properties (e.g. tensile strength). According to various aspects of the present disclosure, certain materials with generally high tensile strengths may be weaved (or otherwise integrated) with the surrounding materials of the garment, thus creating regions of the garment that provide greater compression to a wearer than other regions of the same garment. In particular embodiments, the garment may be constructed or assembled in such a way that these regions may provide compression and support similar to medically relevant taping methods, providing physical benefits such as support, compression, and stability during dynamic movement. In various embodiments, these garment regions may be designed in patterns to accommodate and/or target particular joints, muscles, bones, ligaments, etc., where particular portions of the pattern include higher tensile strengths than others.

For example, consider a scenario where an athlete typically applies tape to his/her feet and ankles prior to physical activity for preventing injuries and generally improving athletic performance. Traditional ankle taping techniques include applying one or more strips of tape around the lower shin for forming a pattern commonly known as an anchor. Continuing with this example, additional tape is typically applied for creating one or more stirrups, which are generally long strips of tape applied to opposite sides of the anchor and wrapped around the bottom of the foot. In this example, the athlete may continue to apply tape around the foot and ankle until adequate compression and support is attained (an inefficient process where tape is frequently removed and discarded).

Aspects of the present disclosure allow for the athlete discussed immediately above to wear a sock, sleeve, or other technical garments, where the worn garment provides benefits similar to athletic taping, without the need to apply tape. As described throughout the present disclosure, portions of the garment may include interweaved materials of varying tensile strengths (and other compressive/tension-resistive properties) for creating particular areas of the garment that, when worn, provide compression and support to the wearer as the particular areas resist tension introduced by the wearer (e.g., the particular areas resist being stretched when worn, thus feeling compressive to the wearer). Embodiments of the exemplary garments discussed herein may employ a wide variety of materials for providing support and compression. Those exemplary materials include, but are not limited to: nylon, natural gum rubber, elastic, cotton, polychloroprene, and/or polyester-spandex.

In various embodiments, the technical garments described herein may be designed to promote normal dynamic movement of joints without altering the biomechanics of regular shock absorption within peripheral joints. By wearing the exemplary garments described herein, wearers/users can protect their joints without altering their biomechanics in a way which may negatively affect other parts of the body thus allowing the technical garments to be used as both a preventative and a rehabilitative technology.

As will be understood from the discussions herein, the portions of the technical garment include substantially high tensile strengths may be designed in various shapes and sizes of varying dimensions similar to that of medically relevant tape that provides compression and support. In various embodiments, the compressive portions may be about 1 inch wide. In some embodiments, the compressive portions may be 0.1 inches to 2.5 inches wide. As will also be understood from the discussions herein, a compression garment may include more than one compressive/resistive region and each region may have the same or different length and width dimensions.

Moreover, the compression garments discussed herein may include any suitable percentage of compressive/resistive materials. In particular embodiments, the surface area of the compression garment described herein may include about 30% compressive and/or resistive materials, where the remainder of the surface area is a base material, such as cotton, wool, or other appropriate technical materials. In some embodiments, the surface area of the compression garment described herein may include about 20% compressive and/or resistive materials. In further embodiments, the surface area of the compression garments discussed herein may include about 2% to 70% compressive and/or resistive materials.

In certain embodiments, the compressive and/or resistive materials, techniques, and patterns included in the socks and sleeves discussed herein may be implemented in any appropriate garment. For example, the compression regions and patterns described herein may be implemented in garments such as shorts, shirts, sleeves and gloves for providing compression and support to body areas such as the knee, hips, shoulders, arms and fingers, respectively.

In various embodiments, the compression regions of the garments that provide support similar to medically relevant taping patterns may be manufactured to include compression levels of varying ranges. For example, a compression garment with regions providing support to the knee may have compression levels ranging from 23-93 mmHg, a compression garment with regions providing support to the ankle may have compression levels ranging from 23-93 mmHg, a compression garment with regions providing support to the elbow may have compression levels ranging from 23-29 mmHg, a compression garment with regions providing support to the shoulder may have compression levels ranging from 11-30 mmHg, and a compression garment with regions providing support to the hip may have compression levels ranging from 74-93 mmHg.

These and other aspects, features, and benefits of the claimed embodiment(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 is a perspective view of an exemplary joint stabilization sock, according to one embodiment of the present disclosure;

FIG. 2 is a front view of an exemplary joint stabilization sock, according to one embodiment of the present disclosure;

FIG. 3 is a rear view of an exemplary joint stabilization sock, according to one embodiment of the present disclosure;

FIG. 4 is a right side view of an exemplary joint stabilization sock, according to one embodiment of the present disclosure;

FIG. 5 is a left side view of an exemplary joint stabilization sock, according to one embodiment of the present disclosure;

FIG. 6 is a top view of an exemplary joint stabilization sock, according to one embodiment of the present disclosure;

FIG. 7 is a bottom view of an exemplary joint stabilization sock, according to one embodiment of the present disclosure;

FIG. 8 is a perspective view of an exemplary joint stabilization elbow sleeve, according to one embodiment of the present disclosure;

FIG. 9 is a front view of an exemplary joint stabilization elbow sleeve, according to one embodiment of the present disclosure;

FIG. 10 is a rear view of an exemplary joint stabilization elbow sleeve, according to one embodiment of the present disclosure;

FIG. 11 is a right side view of an exemplary joint stabilization elbow sleeve, according to one embodiment of the present disclosure;

FIG. 12 is a left side view of an exemplary joint stabilization elbow sleeve, according to one embodiment of the present disclosure;

FIG. 13 is a top view of an exemplary joint stabilization elbow sleeve, according to one embodiment of the present disclosure;

FIG. 14 is a bottom view of an exemplary joint stabilization elbow sleeve, according to one embodiment of the present disclosure;

FIG. 15 is a perspective view of an exemplary joint stabilization knee sleeve, according to one embodiment of the present disclosure;

FIG. 16 is a front view of an exemplary joint stabilization knee sleeve, according to one embodiment of the present disclosure;

FIG. 17 is a rear view of an exemplary joint stabilization knee sleeve, according to one embodiment of the present disclosure;

FIG. 18 is a right side view of an exemplary joint stabilization knee sleeve, according to one embodiment of the present disclosure;

FIG. 19 is a left side view of an exemplary joint stabilization knee sleeve, according to one embodiment of the present disclosure;

FIG. 20 is a top view of an exemplary joint stabilization knee sleeve, according to one embodiment of the present disclosure; and

FIG. 21 is a bottom view of an exemplary joint stabilization knee sleeve, according to one embodiment of the present disclosure.

### DETAILED DESCRIPTION

### Overview

For the purpose of promoting an overview of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

The present disclosure describes embodiments of joint stabilization garments, manufactured from materials with variable material properties. In one embodiment, the garments may include a plurality of portions or regions, where each portion or region includes a particular material with particular properties (e.g. tensile strength). According to various aspects of the present disclosure, certain materials with generally high tensile strengths may be weaved (or otherwise integrated) with the surrounding materials of the garment, thus creating regions of the garment that provide greater compression to a wearer than other regions of the same garment. In particular embodiments, the garment may be constructed or assembled in such a way that these regions may provide compression and support similar to medically relevant taping methods, providing physical benefits such as support, compression, and stability during dynamic movement. In various embodiments, these garment regions may be designed in patterns to accommodate and/or target particular joints, muscles, bones, ligaments, etc., where particular portions of the pattern include higher tensile strengths than others.

For example, consider a scenario where an athlete typically applies tape to his/her feet and ankles prior to physical activity for preventing injuries and generally improving athletic performance. Traditional ankle taping techniques include applying one or more strips of tape around the lower shin for forming a pattern commonly known as an anchor. Continuing with this example, additional tape is typically applied for creating one or more stirrups, which are generally long strips of tape applied to opposite sides of the anchor and wrapped around the bottom of the foot. In this example, the athlete may continue to apply tape around the foot and ankle until adequate compression and support is attained (an inefficient process where tape is frequently removed and discarded).

Aspects of the present disclosure allow for the athlete discussed immediately above to wear a sock, sleeve, or other technical garment, where the worn garment provides benefits similar to athletic taping, without the need to apply tape. As described throughout the present disclosure, portions of the garment may include interweaved materials of varying tensile strengths (and other compressive/tension-resistive properties) for creating particular areas of the garment that, when worn, provide compression and support to the wearer as the particular areas resist tension introduced by the wearer (e.g., the particular areas resist being stretched when worn, thus feeling compressive to the wearer). Embodiments of the exemplary garments discussed herein may employ a wide variety of materials for providing support and compression. Those exemplary materials include, but are not limited to: nylon, natural gum rubber, elastic, cotton, polychloroprene, and polyester-spandex.

In various embodiments, the technical garments described herein may be designed to promote normal dynamic movement of joints without altering the biomechanics of regular shock absorption within peripheral joints. By wearing the exemplary garments described herein, wearers/users can protect their joints without altering their biomechanics in a way which may negatively affect other parts of the body thus allowing the technical garments to be used as both a preventative and rehabilitative technology.

As will be understood from the discussions herein, the portions of the technical garment including substantially high tensile strengths may be designed in various shapes and sizes of varying dimensions. In various embodiments, the compressive portions may be about 1 inch wide. In some embodiments, the compressive portions may be 0.1 inches to 2.5 inches wide. As will also be understood from the discussions herein, a compression garment may include more than one compressive/resistive region and each region may have the same or different length and width dimensions.

Moreover, the compression garments discussed herein may include any suitable percentage of compressive/resistive materials. In particular embodiments, the surface area of the compression garment described herein may include about 30% compressive and/or resistive materials, where the remainder of the surface area is a base material, such as cotton, wool, or other appropriate technical materials. In some embodiments, the surface area of the compression garment described herein may include about 20% compressive and/or resistive materials. In further embodiments, the surface area of the compression garments discussed herein may include about 2% to 70% compressive and/or resistive materials.

In certain embodiments, the compressive and/or resistive materials, techniques, and patterns included in the socks and sleeves discussed herein may be implemented in any appropriate garment. For example, the compression regions and patterns described herein may be implemented in shorts, shirts, and gloves for providing compression and support to body areas such as hips, shoulders, and fingers, respectively.

These and other aspects, features, and benefits of the claimed embodiment(s) will become apparent from the following detailed written description of the preferred embodiments and aspects taken in conjunction with the following drawings, although variations and modifications thereto may be effected without departing from the spirit and scope of the novel concepts of the disclosure.

### Exemplary Embodiments

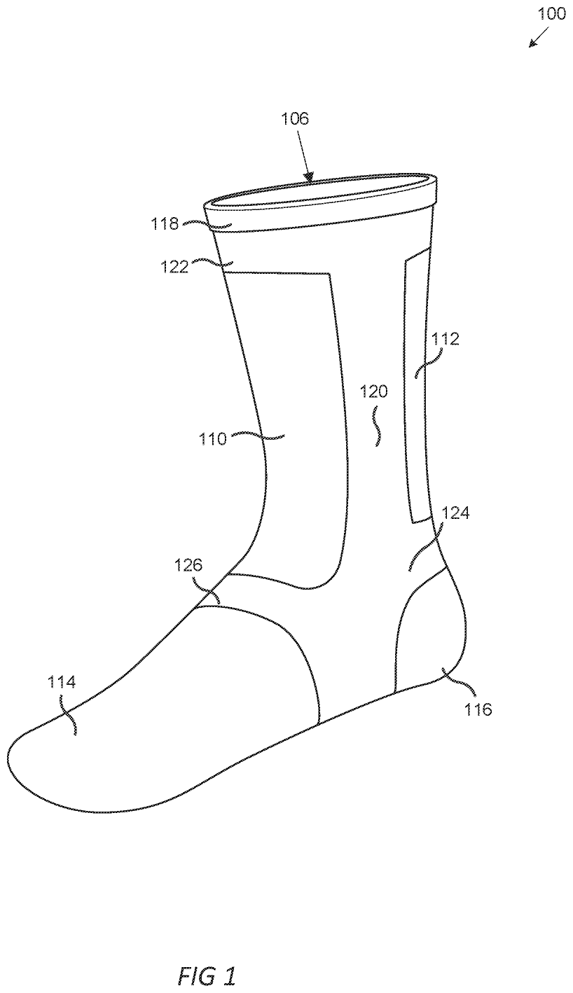

Referring now to the figures, for the purposes of example and explanation of the disclosed garment(s), reference is made to FIG. 1, which illustrates one embodiment of a joint stabilization sock 100. As will be understood and appreciated, the exemplary joint stabilization sock 100 shown in FIG. 1 represents merely one approach or embodiment of the joint stabilization sock 100, and other variations and embodiments are described herein.

FIG.1 illustrates a perspective view of the exemplary joint stabilization sock 100, according to one embodiment of the present disclosure. Generally, the sock 100 shown in the present embodiment can be worn on a foot and leg of a wearer. During manufacturing, the sock may be constructed with a left or right orientation for accommodating both left and right feet, providing an improved fit for athletic use. In various embodiments, the sock 100 includes an opening 106 that may allow the user to wear the garment by placing his/her foot into the opening 106. In some embodiments, the joint stabilization sock 100 may be manufactured to be compatibly worn on either foot. In various embodiments, the joint stabilization sock 100 can be manufactured and constructed from any suitable raw material(s) (e.g., cotton, synthetic yarn, man-made fibers, polymers and elastic fibers, nylon, natural gum rubber, elastic, cotton, polychloroprene, and/or polyester-spandex, etc.) by a knitting process, or the like.

As shown in the present embodiment, the joint stabilization sock 100 includes separate or distinct regions 114(wearer's toes), regions 126,124 (wearer's foot and/or ankle), region 116 (wearer's heel) and regions110, 112, 120, 122 (wearer's leg). These regions include materials of a various tensile strengths interwoven or otherwise integrated into a base material of the garment for providing compressive and/or resistive forces to the wearer, where the compressive and/or resistive forces are similar to those achieved via athletic taping practices. In one embodiment, the regions of the exemplary joint stabilization sock 100 that include the materials of various tensile strengths may be designed in various shapes and dimensions, as appropriate. For example, in some embodiments, particular regions of the sock may range from about 0.1 inches to 2.5 inches in width (e.g., similar to the dimensions of athletic tape). In certain embodiments, these regions may be graduated or extended along the length of the sock 100, from a distal portion to a proximal portion, so as to enhance stability, provide arch support, and provide resistance to inversion.

Changes or transitions between regions of varying tensile strengths in the stabilization sock 100 may be gradual, so as to provide a smooth transition between regions, which not only enhances comfort but also reduces the risk of the tourniquet effect. In various embodiments, changes or transitions between regions of varying tensile strength may be abrupt, opposed to gradual.

In particular embodiments, the sock regions 110, 112, 114, 116 and 118 include a base material manufactured from polyester spandex, breathable cotton, a combination thereof, or any suitable material. In various embodiments, sock regions 122 and 120, which may integrate with the lower regions 126 and 124, provide compression and support similar to taping patterns that wrap around the bottom of the foot for preventing injuries such as inversion sprains. The materials of regions 122 and 120 may be different from regions 126 and 124, or, in some embodiments, the materials may be substantially similar. In various embodiments, regions 122 and 120 may include the synthetic rubber material polychloroprene, along with nylon band and natural gum rubber materials. In particular embodiments, both regions 122 and 118 extend around the top portion of the sock and may provide compression for maintaining an upright orientation of the sock on the wearer.

In one embodiment, region 126 provides compression and support similar to a taping pattern that wraps around the arch of the foot, the top of the foot where the foot and ankle meet, and the rear portion of the arch of the foot. In particular embodiments, the region 126 is designed to provide stability, arch support, and compression/resistance for preventing inversion sprains. In some embodiments, the materials of region 126 may include elastic cotton, nylon band and/or natural gum rubber or any other materials discussed herein. In one embodiment, region 124 provides compression and support similar to a taping pattern that wraps around the Achilles tendon and the front of the arch of the foot providing stability to both areas. The materials of region 124 may include elastic cotton, nylon band and natural gum rubber.

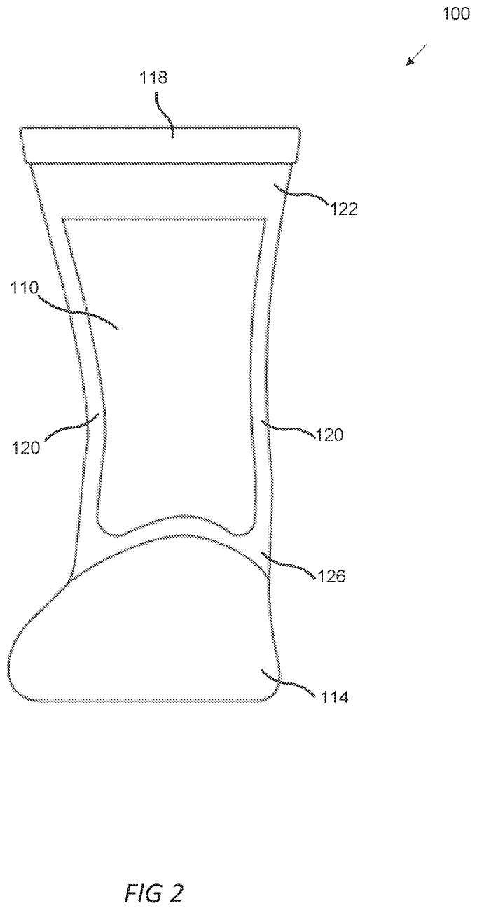

In various embodiments, region 118 forms a cuff of the sock 100 and generally is ribbed so it clings to the leg of the wearer. In one embodiment, region 114 includes sole and toe portions of the sock. In some embodiments, region 116 includes the heel portion of the sock. The corresponding portions of the sock 100 of FIGS. 2-7 include the same reference numbers as discussed in association with FIG. 1.

FIG. 2 illustrates a front view of the exemplary joint stabilization sock 100, according to one embodiment of the present disclosure. In one embodiment, the upper portion of region 114 meets and merges, or integrates and interweaves, with region 126. In various embodiments, region 126 generates compression and support similar to the medical taping pattern that provides arch support and resistance to inversion sprains. In the present embodiment, region 126 wraps around a portion of the sock 100 which may cover where the foot and ankle meet.

In various embodiments, region 114 can be manufactured with a stockinette, kitchener, stocking, and/or any appropriate stitch so that it is smooth and comfortable and can fit inside of a shoe. In some embodiments, the region 114 includes an area for the toes (individually or collectively) of the wearer. Region 114 may have various suitable designs, including but not limited to turkish toe, wedge toe, start toe and/or short row toe.

In various embodiments, region 110 of the sock 100 extends upwardly from above the ankle and is horizontally positioned along the shin of the wearer when worn. In various embodiments, region 110 tapers outwardly and merges with region 120. In various embodiments, region 110 also extends upwardly and merges with region 122. It will be recognized that sock 100 can include the same material components as described in association with the other figures herein.

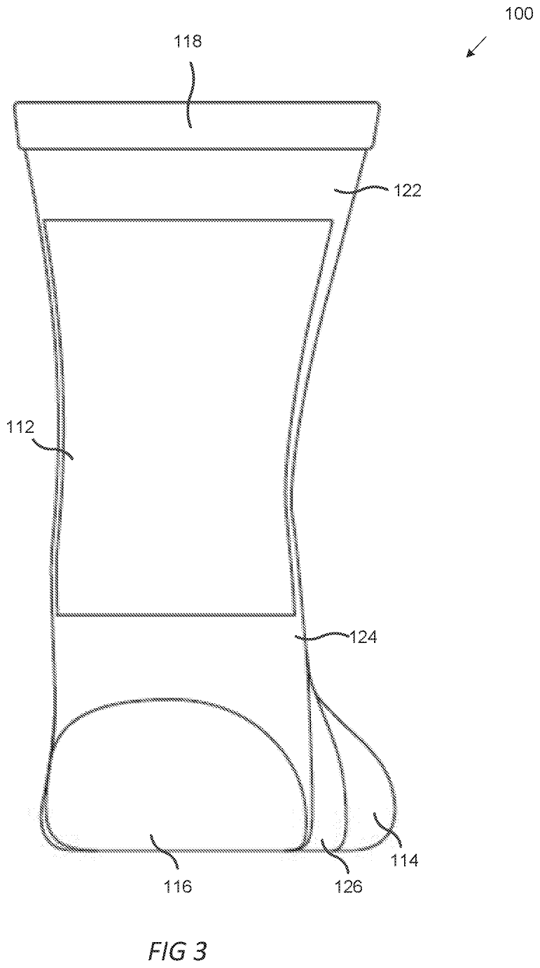

FIG. 3 illustrates a rear view of the exemplary joint stabilization sock 100, according to one embodiment of the present disclosure. As shown, region 112 of the sock 100 includes a top end, a bottom end and two sides. In the embodiment shown in FIG. 3, region 112 covers the user's/wearer's calf and may be constructed with stretchable and breathable materials, such as polyester spandex and/or cotton. As shown in the present embodiment, the top end of region 112 merges with region 122. In various embodiments, the bottom end of region 112 merges with region 124.

In various embodiments, region 116 covers the wearer's heel. Region 116 may integrate with region 126. In some embodiments, region 114 may cover the wearer's toes.

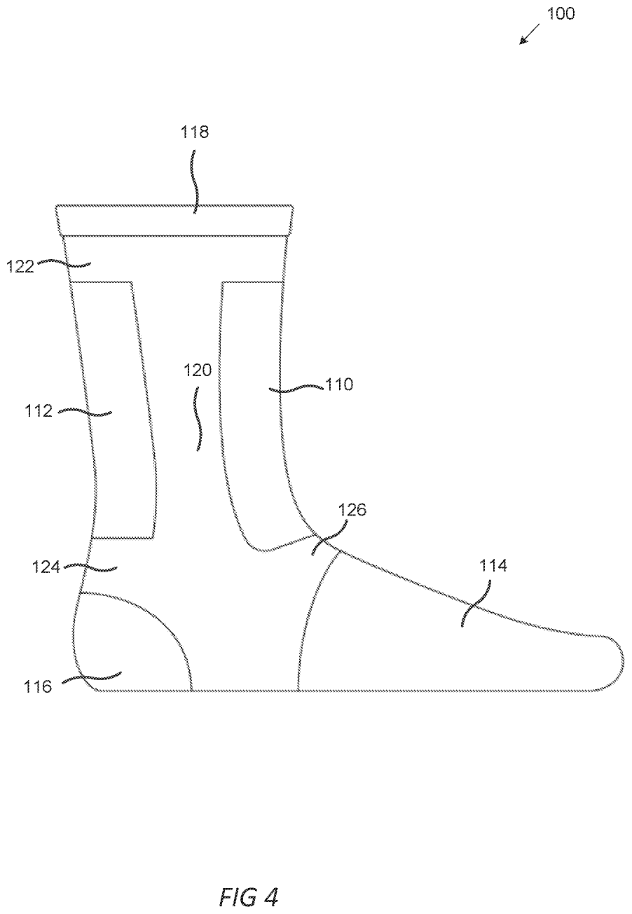

FIG. 4 illustrates the right side view of the exemplary joint stabilization sock 100, according to one embodiment of the present disclosure. As discussed herein, in various embodiments, the sock 100 includes separate or distinct regions which include areas for the wearer's toe, foot and/or ankle, heel and leg integrated as a unitary garment. In one embodiment, the compression patterns of regions 124 and 126 may begin on the sole of the sock 100 and continue upwards around the top of the sock 100 and the rear of the sock 100. In particular embodiments, the region 126 is designed to extend around the top of a wearer's foot for providing arch support and compression benefits similar to those achieved via medical taping practices, which include wrapping tape around the feet and ankles. In certain embodiments, region 126 is constructed from stretchable, highly resilient elastic materials such as, elastic cotton, nylon band and/or natural gum rubber. In particular embodiments, the region 124 include materials with high tensile strength properties for resisting potentially harmful forces or movements of the wearer, particularly undesired movements in the ankles and/or Achilles tendon. In one embodiment, region 124 is constructed from the same materials or substantially similar materials as region 126. In various embodiments, the taping pattern of region 120 begins at the top of the shin and intersects with region 124 and 126 wraps around the sole and/or bottom of the sock 100. In one embodiment, region 120 is designed to provide compression and support to a wearer's foot and leg for preventing injuries such as sprains and the like. In various embodiments, region 120 is constructed from stretchable elastic materials such as nylon band, natural gum rubber and/or polychloroprene. As shown in FIG. 4, regions 112 and 110 include a top end, a bottom end and two sides.

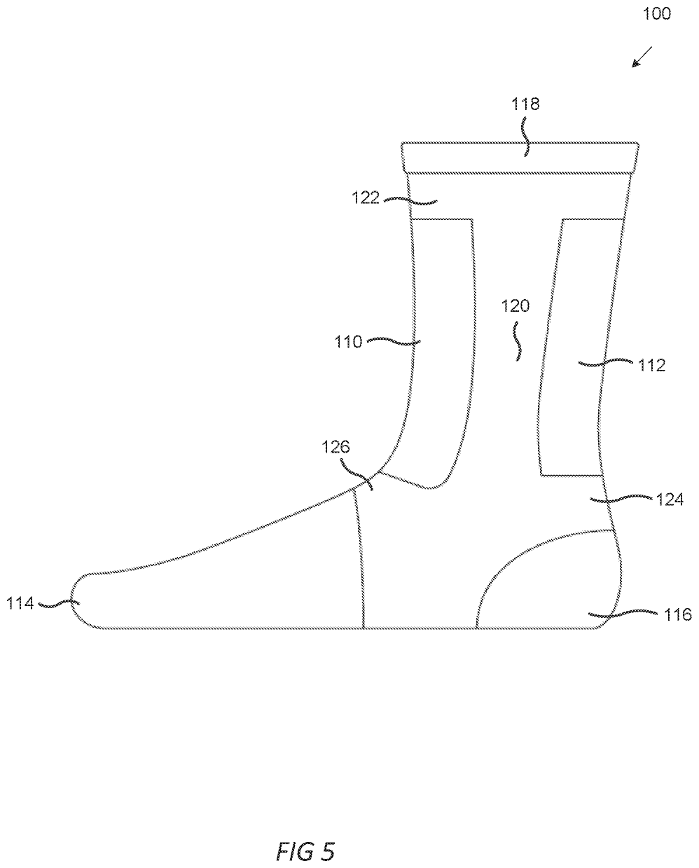

FIG. 5 illustrates the left side view of the exemplary joint stabilization sock 100, according to one embodiment of the present disclosure. In one embodiment, the materials and regions of the left side view of sock 100 are substantially similar to those described in association with FIG. 4.

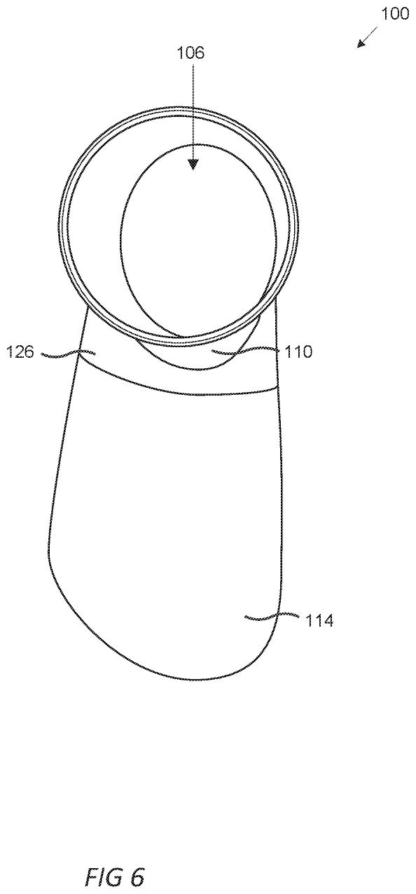

FIG. 6 illustrates a top view of the exemplary joint stabilization sock 100, according to one embodiment of the present disclosure. As shown in the present embodiment, region 114 may be made of the base materials polyester spandex and/or breathable cotton. Region 126, in various embodiments, provides compression and support similar to a medical taping pattern that wraps around the top of a wear's foot. In various embodiments, region 110 is included in the body section of the sock 100, as described above in association with the discussion of FIGS. 1-5. In some embodiments, region 106 is an interior portion. The materials for region 106 are substantially similar to other materials or regions described herein.

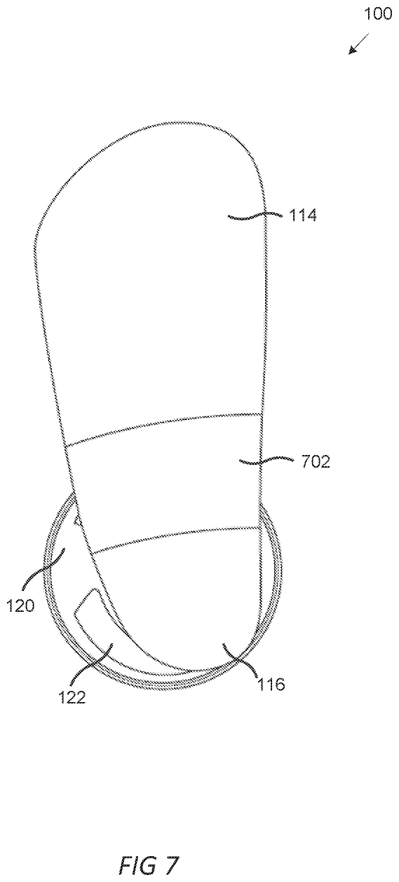

FIG. 7 illustrates a bottom view of the exemplary joint stabilization sock 100, according to one embodiment of the present disclosure. As depicted, the sole of foot includes the region 114 which, as described above, may be manufactured from materials such as polyester spandex and/or breathable cotton. As shown in the present embodiment, region 702 extends across the bottom of the sock 100. According to various aspects of the present disclosure, the region 702 may include a tensile strength greater than adjacent sock regions and/or portions (114 and 116), such that the greater tensile strength may provide compression to the bottom of a wearer's foot (e.g., to support the wearer's plantar fascia ligament, prevent tendonitis, or generally provide support for preventing sprains and rolls). In some embodiments, region 702 may be a combination or integration of regions 124 and 126 (not shown), which are described above in association with FIGS. 1-6.

In one embodiment, region 116 covers the heel portion of sock 100. In various embodiments, the heel may be manufactured with different stitching and/or knitting techniques. Different types of techniques include but not limited to short row, heel flap and gusset, and Dutch heel. Regions 120 and 122 are substantially similar to the corresponding regions described above in FIGS. 1-5.

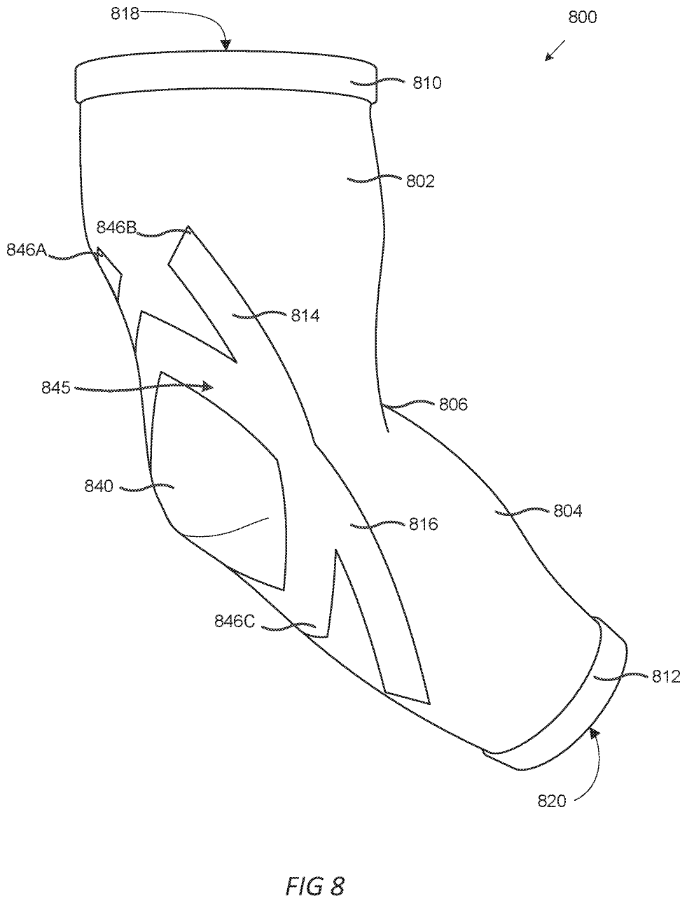

Turning now to FIG. 8, a prospective view of an exemplary elbow joint stabilization sleeve 800 is shown, according to one aspect of the present disclosure. Generally, the elbow sleeve 800 includes upper 802, middle 806, and lower 804 portions of the sleeve, where each portion may include varying properties for providing comfort and support to the wearer. In one embodiment, the upper portion 802 may be designed to cover the upper arm of a wearer, where the upper portion 802 of the sleeve 800 may extend from the elbow to the bicep. In various embodiments, the middle portion 806 of the sleeve 800 may be designed to cover the elbow of a wearer. In particular embodiments, the lower portion 804 of the sleeve 800 may be designed to cover the forearm of a wearer, and may extend from the elbow to hand.

In various embodiments, the elbow stabilization sleeve 800 may include any suitable shape (e.g. generally tube-shaped). In some embodiments, the upper portion 802 includes an upper sleeve opening 818 and the lower portion 804 includes a lower sleeve opening 820. In many embodiments, the user may wear the garment by placing his/her hand first into the sleeve opening 818 and sliding the sleeve to his/her elbow until his/her hand extends from the opening 820.

In some embodiments, the elbow joint stabilization sleeve 800 can be manufactured and constructed from any suitable raw material(s) (e.g., cotton, synthetic yarn, man-made fibers, polymers and elastic fibers, nylon, natural gum rubber, elastic, cotton, polychloroprene, and/or polyester-spandex, etc.) by a knitting process, or the like. In various embodiments, regions (814,816, 845) of the elbow sleeve 800 may be manufactured to have compression levels ranging from 23-29 mmHg.

As shown in the present embodiment, the elbow joint stabilization sleeve 800 includes separate or distinct regions located from the middle portion 814 (e.g. elbow) extending downwards towards the lower portion 816 (e.g. back of the forearm). These regions (814 and 816) include materials of a various tensile strengths interwoven or otherwise integrated into a base material of the garment for providing compressive and/or resistive forces to the wearer around these regions, where the compressive and/or resistive forces are similar to those achieved via athletic taping practices. In one embodiment, the regions (814 and 816) of the exemplary elbow joint stabilization sleeve 800 that include the materials of various tensile strengths may be designed in various shapes and dimensions, as appropriate. In certain embodiments, these regions (814 and 816) may be graduated or extended along the length of the elbow, from a distal posterior portion to a proximal posterior portion, so as to enhance stability, provide ligament support, and provide resistance to inversion.

Continuing with FIG. 8, the regions 814 and 816 discussed immediately above may be included in a compression region 845 for supporting the wearer's elbow. As shown in the present embodiment, the compression region 845 includes a geometric configuration characterized by intersecting arches, where the intersecting arches may include substantially high tensile strengths, and the arches form a substantially diamond-shaped center 840. In particular embodiments, the compression region 845 includes four intersecting arches, where each arch is for targeted compression on a particular portion of the wearer's body (e.g., the wearer's elbow covered by the substantially diamond-shaped center 840). In this embodiment and others, at least two opposing arches extend from separate locations, 846A and 846B, to join or integrate at a point 846C located at an end of the arches. According to various aspects of the present disclosure, the arches in this configuration are “opposing” such that they are located on opposite sides of a targeted body region (e.g., the wearer's elbow) and each separately provides compression on opposite sides of the wearer's elbow to prevent the wearer's elbow from moving or dislocating beyond the arches. As shown in the present embodiment, the configuration of the compression region 845 allows for the wearer's elbow to be maintained medially between these regions, where the diamond-shaped center 840 covers the wearer's elbow.

Changes or transitions between regions of varying tensile strengths in the elbow joint stabilization sleeve 800 may be gradual, so as to provide a smooth transition between regions, which may not only enhances comfort but may also reduce the risk of the tourniquet effect. In some embodiments, changes or transitions between regions of varying tensile strengths may be abrupt, opposed to gradual.

In particular embodiments, the elbow sleeve regions 802, 804, 806, 810 and 812 include a base material manufactured from polyester spandex, breathable cotton, a combination thereof, or any suitable material. In some embodiments, elbow sleeve region 814, which may integrate with the lower region 816, may provide compression and support similar to taping patterns that wrap around a portion of the elbow and extend downward towards the posterior portion of the forearm for preventing injuries such as dislocations, ligament tears, strains and/or sprains. In various embodiments, two or more taping patterns may be similar to that of the taping patterns of the sock 100 (FIG. 1). The materials of region 814 may be different from region 816 or, in some embodiments, the materials may be substantially similar. In various embodiments, the regions 814 and 816 may include one or more materials similar to the joint stabilizing sock 100 (FIG. 1).

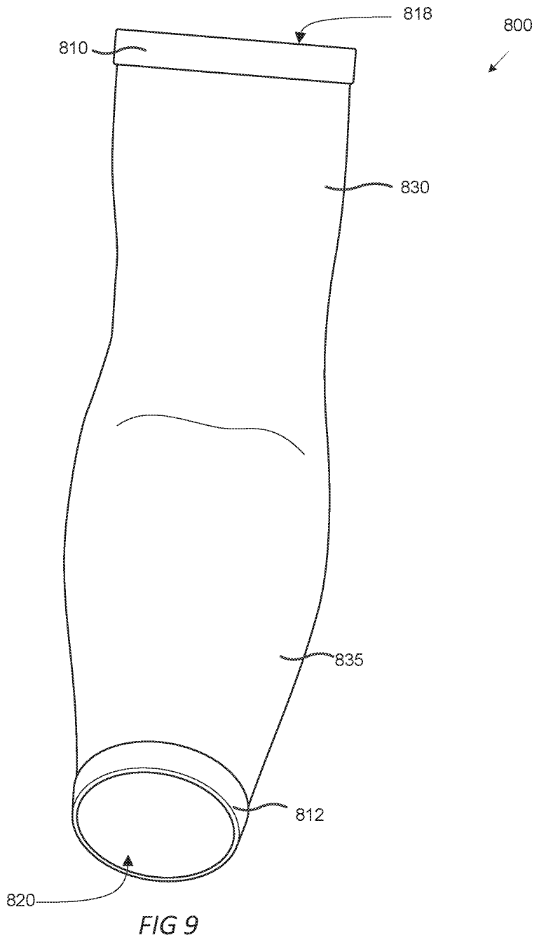

In one embodiment, regions 810 and 812 form the cuffs of elbow sleeve 800 and generally are ribbed so the regions 810 and 812 cling to the either the upper arm or forearm of a wearer. The corresponding portions of the elbow sleeve 800 of FIGS. 9-14 include the same reference numerals as shown in FIG.8.

FIG. 9 illustrates a front view of the exemplary joint stabilization elbow sleeve 800, according to one embodiment of the present disclosure. As shown, regions 830 and 835 include a base material manufactured from polyester spandex, breathable cotton, a combination thereof, or any suitable material. In one embodiment, a wearer may wear the garment by placing a hand first into the sleeve opening 818 and sliding the sleeve past the wearer's elbow until his/her hand extends through the opening 820.

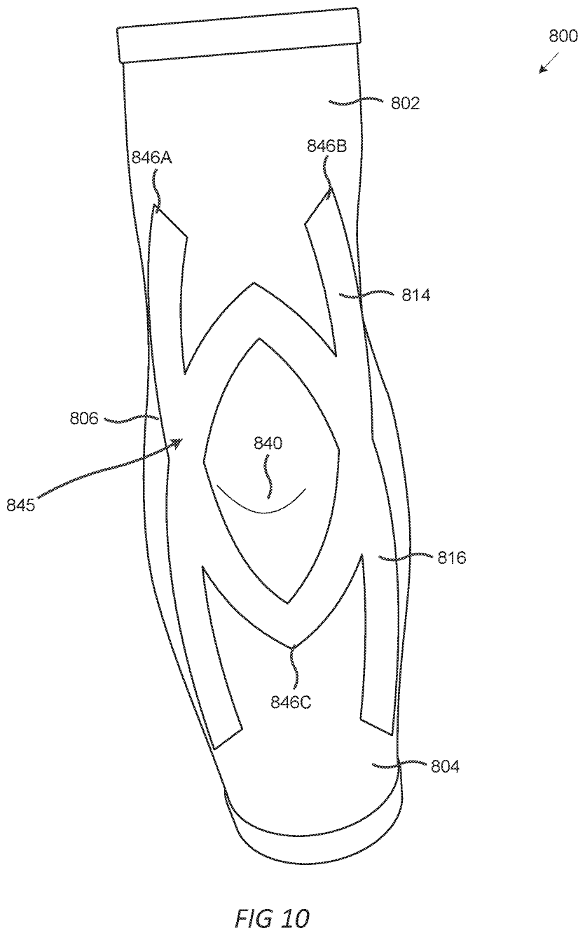

FIG. 10 illustrates a rear view of the exemplary joint stabilization elbow sleeve 800, according to one embodiment of the present disclosure. Generally, the elbow sleeve 800 includes upper 802, middle 806 and lower 804 portions. In some embodiments, the compression pattern of region 814 may begin from the upper portion of the garment and continue downwards merging with region 816 towards the lower portion of the garment. In some embodiments, region 814 meets and merges, or integrates and interweaves with region 816.

In various embodiments, regions 814 and 816 generate compression and support similar to a medical taping pattern that provides joint support, resistance to ligament tears, and prevents dislocation of the elbow joint. In the present embodiment, region 814 wraps around a portion of the elbow sleeve 800 merging with region 816 which may cover where the elbow and posterior forearm meet. In some embodiments, regions 814 and 816 include materials with high tensile strength properties for resisting potentially harmful forces or movements of the wearer, particularly undesired movements in the elbow and associated ligaments. As discussed in association with FIG.8, in various embodiments, the compression region 845 may include a geometric configuration characterized by intersecting arches where the arches may form a substantially diamond shaped center 840. In some embodiments, the compression region 845 may include four or more intersecting arches. In particular embodiments, at least two opposing arches extend from separate locations, 846A and 846B, to join or integrate at a point 846C located at an end of the arches. In some embodiments, the compression regions may form any suitable shape, such as substantially oval, circular, or rectangular.

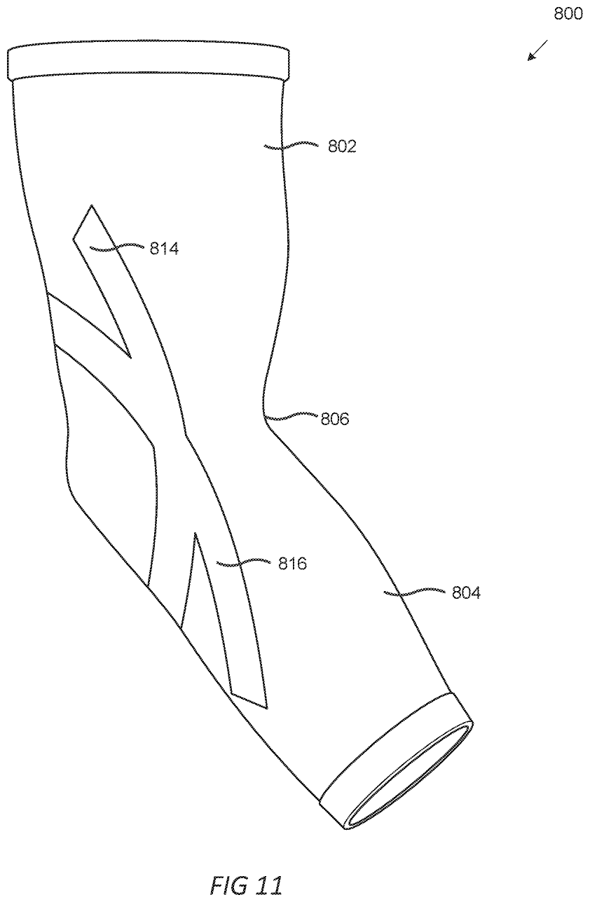

FIG. 11 illustrates the right side view of the exemplary joint stabilization elbow sleeve 800, according to one embodiment of the present disclosure. The elbow sleeve 800 includes an upper 802, middle 806 and lower 804 portion integrated as a unitary garment. In one embodiment, the compression pattern of region 814 may begin from the upper portion 802 of the garment and continue downwards merging with region 816 towards the lower portion 804 of the garment. In particular embodiments, the regions 814 and 816 are designed to extend downwards from the elbow of the wearer towards the posterior forearm for providing support and compression benefits similar to those achieved via medical taping practices that include wrapping tape around the elbow.

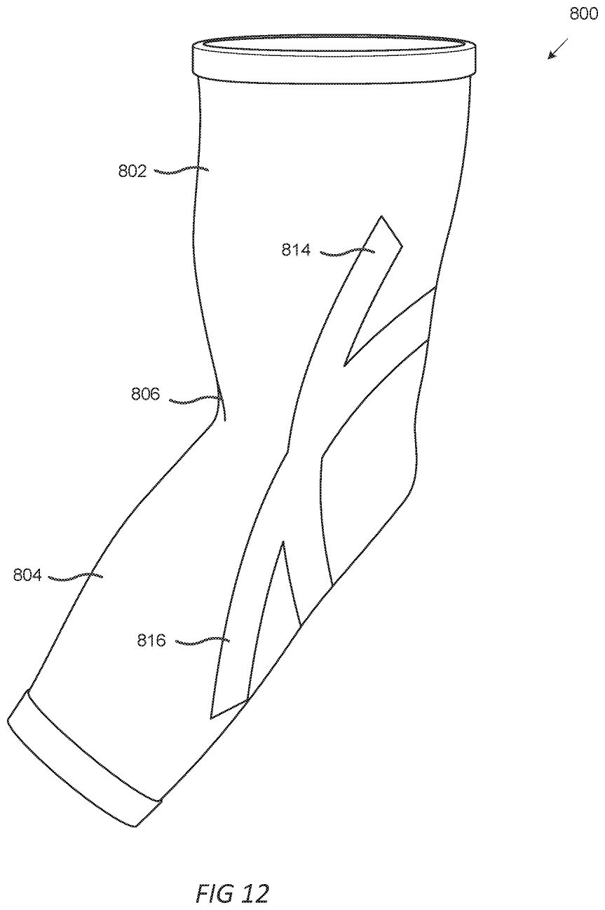

FIG. 12 illustrates the left side view of the exemplary joint stabilization elbow sleeve 800, according to one embodiment of the present disclosure. In at least one embodiment, the materials and regions of the left side view of elbow sleeve 800 are substantially similar to those described in association with FIG. 11.

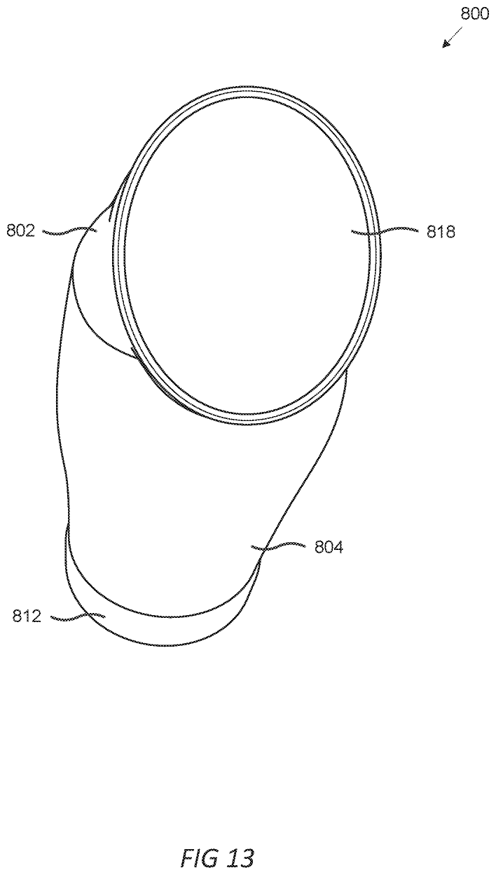

FIG. 13 illustrates a top view of the exemplary joint stabilization elbow sleeve 800, according to one embodiment of the present disclosure. As shown in the present embodiment, the upper 802, middle (not shown) and lower 804 potion include regions made of the base materials polyester spandex and/or breathable cotton. In some embodiments the upper portion 802 includes an opening 818 for inserting the arm of the wearer into the garment and sliding the sleeve upwards until placed on the elbow.

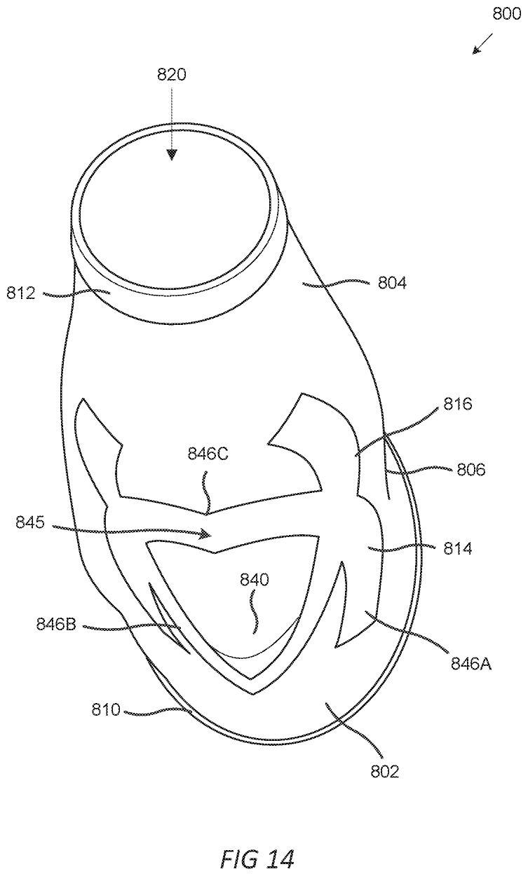

FIG. 14 illustrates a bottom view of the exemplary joint stabilization elbow sleeve 800, according to one embodiment of the present disclosure. As shown in the present embodiment, the lower 804, middle 806 and upper 802 portions of the sleeve include regions 812 and 810, made of the base materials polyester spandex and/or breathable cotton. In some embodiments, the lower portion 804 includes an opening 820 to which the hand of the wearer protrudes. In particular embodiments, the regions 814 and 816 are designed to extend downwards from the elbow of the wearer towards the posterior forearm for providing support and compression benefits similar to those achieved via medical taping practices that include wrapping tape around the elbow.

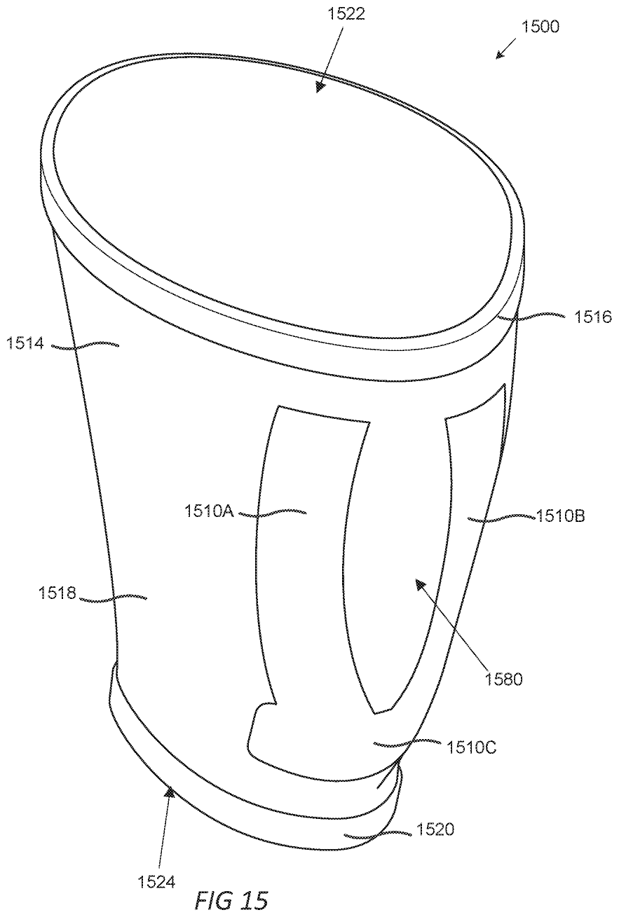

FIG. 15 illustrates a prospective view of an exemplary knee joint stabilization sleeve 1500. Generally, the knee sleeve 1500 includes an upper 1514 and lower 1518 portion. In various embodiments, the knee stabilization sleeve may be of any suitable shape (e.g. generally tube-shaped). In some embodiments, the upper portion 1514 includes an upper sleeve opening 1522 and the lower portion 1518 includes a lower sleeve opening 1524. In various embodiments, the user may wear the garment by placing a foot first into the opening 1522 and sliding the sleeve onto the knee until the user's foot extends from the opening 1524. In some embodiments, the knee joint stabilization sleeve 1500 can be manufactured and constructed from any suitable raw material(s) (e.g., cotton, synthetic yarn, man-made fibers, polymers and elastic fibers, nylon, natural gum rubber, elastic, cotton, polychloroprene, and/or polyester-spandex, etc.) by a knitting process, or the like. In various embodiments, regions 1510(A-C) of the knee sleeve 1500 may be manufactured to have compression levels ranging from 23-93 mmHg.

As shown in the present embodiment, the knee joint stabilization sleeve 1500 includes separate or distinct compression regions (1510A, 1510B, and 1510C) located near the base of the knee joint extending upwards towards the base of the thigh. According to various aspects of the present disclosure, and similar to the compression region 845 discussed above in association with FIG. 8, the compression regions 1510A, 1510B, and 1510C, may be part of a compression region 1580 designed in a particular pattern for providing support to the wearer's knee. In the present embodiment, the compression regions 1510A, 1510B, and 1510C, form a substantially-wishboned shape where the compression regions 1510A and 1510B extend upward (like prongs) from the compression region 1510C covering at least a portion of the knee. In particular embodiments, the compression regions 1510A, 1510B, and 1510C provide semi-opposing compression to a particular portion of the wearer's body covered by the sleeve 1500.

For example, a runner not wearing the exemplary sleeve 1500 may experience a downward force on his/her knee while running. In this example, if the runner were wearing the exemplary sleeve 1500, the compression region 1510C may be just below or partially covering the runner's knee cap (similar to a cradle) and the compression regions 1510A and 1510B may compress upward along the sides of the runner's knee cap and the length of the wearer's thigh. Thus, as the runner's knee experiences downward force, the compression region 1510C resists the downward force via its own compressive properties in combination with the compressive properties of the upwardly extending compression regions 1510A and 1510B. These compression regions may include materials of various tensile strengths interwoven or otherwise integrated into a base material of the garment for providing compressive and/or resistive forces to the wearer around these regions, where the compressive and/or resistive forces are similar to those achieved via athletic taping practices. In one embodiment, the regions 1510A, 1510B, and 1510C of the exemplary knee joint stabilization sleeve 1500 that include the materials of various tensile strengths may be designed in various shapes and dimensions, as appropriate. In certain embodiments, these compression regions may be graduated or extended along the length of the knee, from a distal portion to a proximal portion, so as to enhance stability, provide ligament support, and provide resistance to inversion.

Changes or transitions between regions of varying tensile strengths in the knee joint stabilization sleeve 1500 may be gradual, so as to provide a smooth transition between region, which not only enhances comfort but also reduces the risk of the tourniquet effect. In various embodiments, changes or transitions between regions of varying tensile strengths in the knee joint stabilization sleeve 1500 may be abrupt, opposed to gradual.

In particular embodiments, the knee sleeve regions 1514, 1516, 1518 and 1520 include a base material manufactured from polyester spandex, breathable cotton, a combination thereof, or any suitable material. The wishbone-shaped pattern of the compression regions 1510A, 1510B,and 1510C may provide compression and support similar to taping patterns that wrap around the base of the knee and extend upward towards the thigh for preventing injuries such as dislocations, ligament and/or meniscus tears. In various embodiments, two or more taping patterns may be similar to that of the taping patterns of the sock 100 (FIG. 1). The materials of regions 1510A and 1510B may be different from region 1510C or, in some embodiments, the materials may be substantially similar. In various embodiments, the regions 1510A-C may include one or more materials similar to the joint stabilizing sock 100 (FIG. 1).

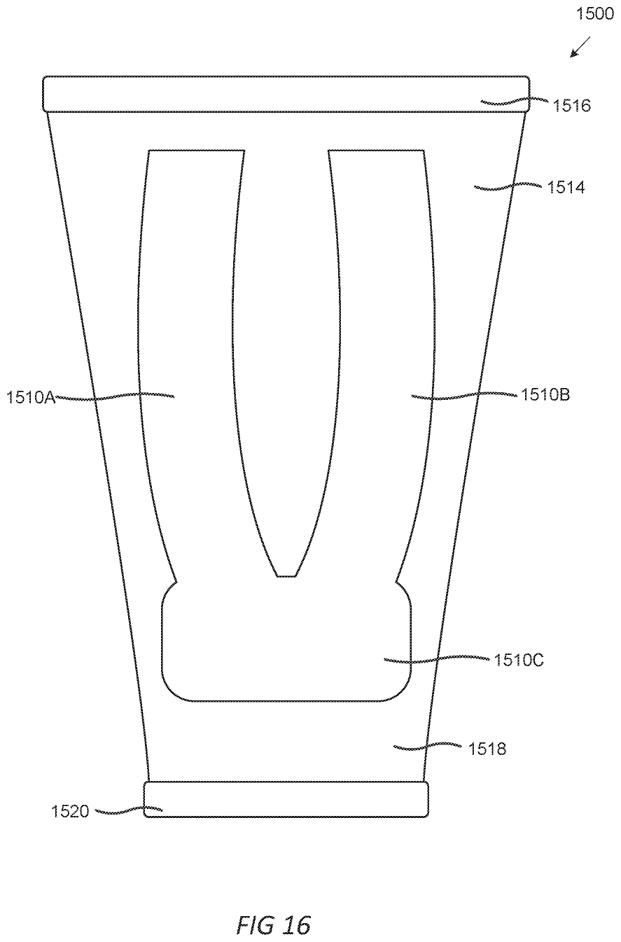

In one embodiment, regions 1516 and 1520 form the cuffs of knee sleeve 1500 and generally are ribbed so the knee sleeve 1500 clings to the either the lower thigh or the base of the knee of a wearer. The corresponding portions of the knee sleeve 1500 of FIGS. 16-21 will include the same reference numerals as FIG. 15.

FIG. 16 illustrates a front view of the exemplary joint stabilization knee sleeve 1500, according to one embodiment of the present disclosure. In some embodiments, the lower portions of regions 1510A and 1510B meet and merge, or integrate and interweave, with the compression region 1510C. In various embodiments, regions 1510A, 1510B, and 1510C generate compression and support similar to the medical taping pattern that provides joint support, resistance to ligament and/or meniscus tears, prevent movement of the knee cap, and prevent dislocation of the knee joint. In the present embodiment, regions 1510A and 1510B wrap around a portion of the knee sleeve 1500 merging with region 1510C, which may cover a portion of the leg where the knee and upper thigh meet. In some embodiments, the regions 1510A, 1510B, and 1510C include materials with high tensile strength properties for resisting potentially harmful forces or movements of the wearer, particularly undesired movements in the knee and associated ligaments.

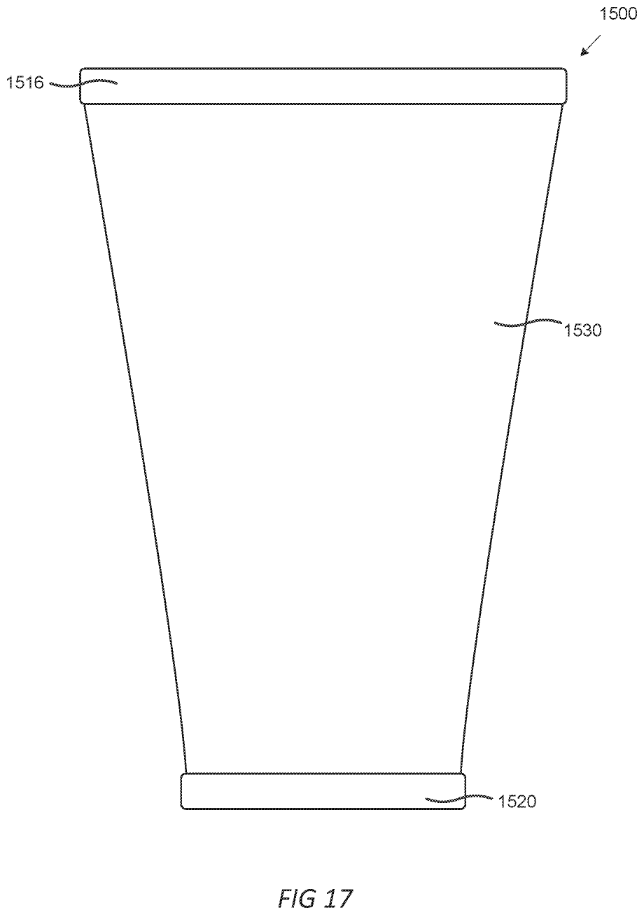

FIG. 17 illustrates a rear view of the exemplary joint stabilization knee sleeve 1500, according to one embodiment of the present disclosure. As shown, region 1530 includes the rear section of the knee sleeve 1500. In some embodiments, region 1530 includes a base material manufactured from polyester spandex, breathable cotton, a combination thereof, or any suitable material.

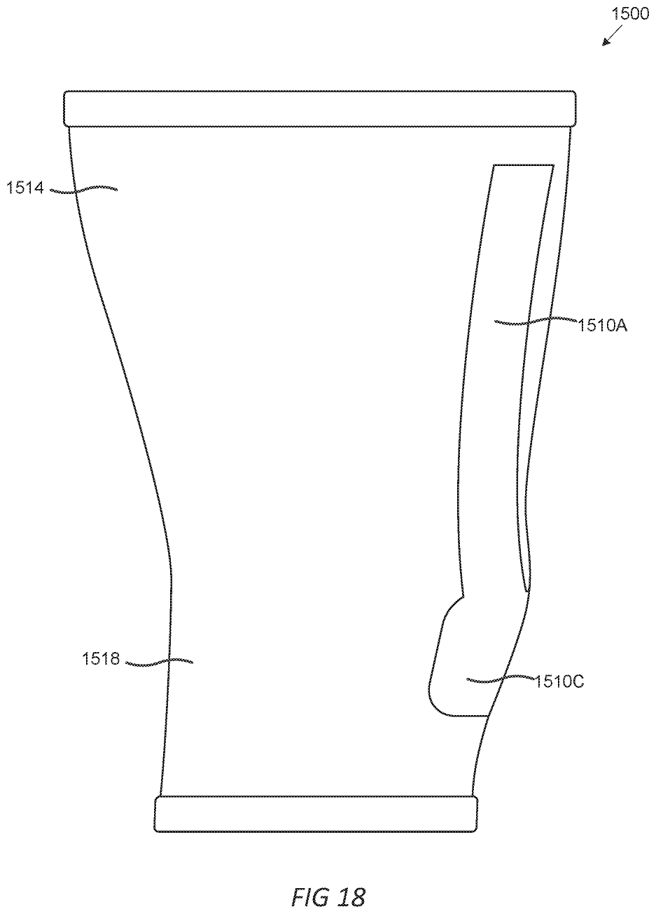

FIG. 18 illustrates the right side view of the exemplary joint stabilization knee sleeve 1500, according to one embodiment of the present disclosure. The knee sleeve 1500 includes an upper 1514 and lower 1518 portion integrated as a unitary garment. In one embodiment, the compression pattern of region 1510A may begin from the upper portion of the garment and continue downwards merging with region 1510C towards the lower portion of the garment. In particular embodiments, the regions 1510A and 1510B are designed to extend downwards from the upper thigh of the wearer towards the knee for providing support and compression benefits similar to those achieved via medical taping practices that include wrapping tape around the knee and/or thigh.

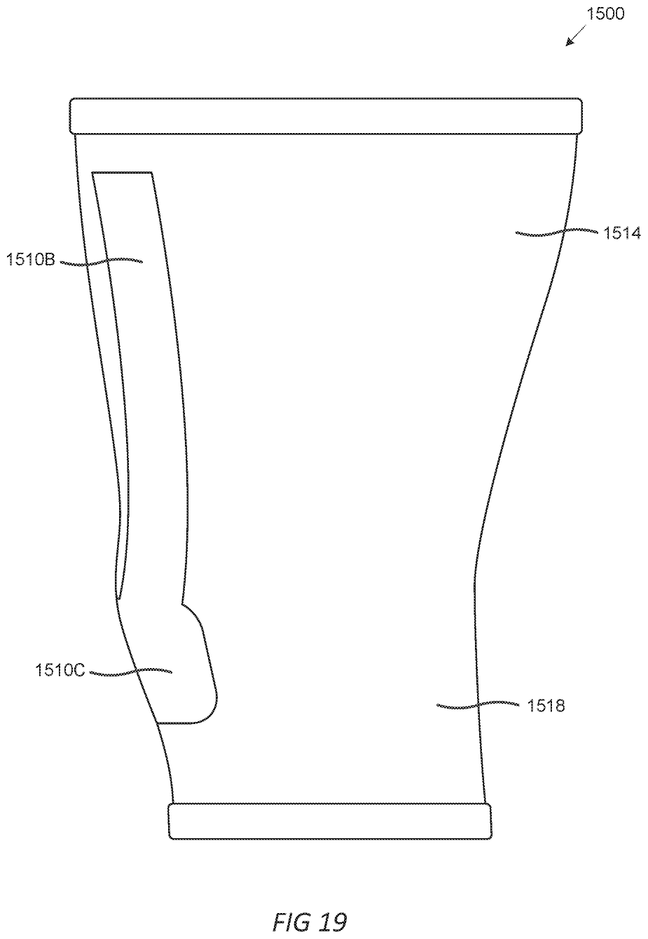

FIG. 19 illustrates the left side view of the exemplary joint stabilization knee sleeve 1500, according to one embodiment of the present disclosure. In some embodiments, the materials and regions of the left side view of knee sleeve 1500 are substantially similar to those described in association with FIG. 18.

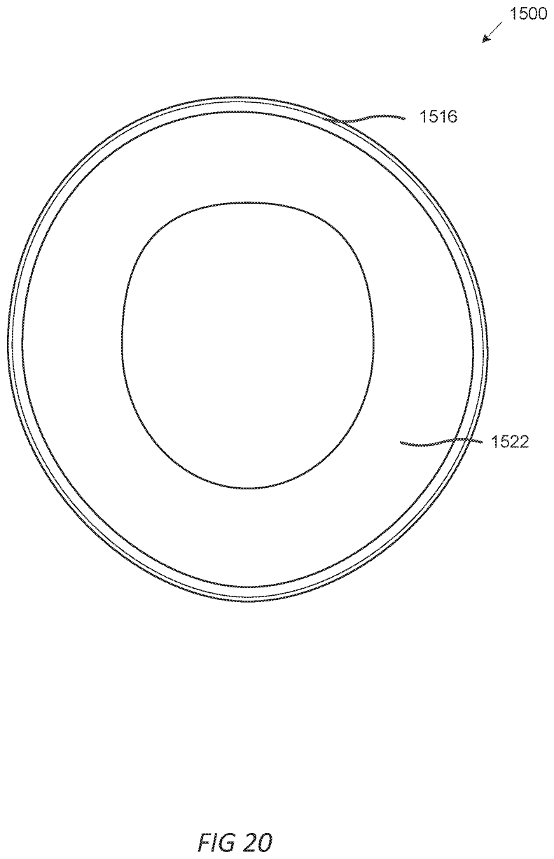

FIG. 20 illustrates a top view of the exemplary joint stabilization knee sleeve 1500, according to one embodiment of the present disclosure. As shown in the present embodiment, the region 1516 may include materials such as polyester spandex and/or breathable cotton. In some embodiments, the upper portion includes a knee opening 1522 for inserting a leg of a wearer into the garment.

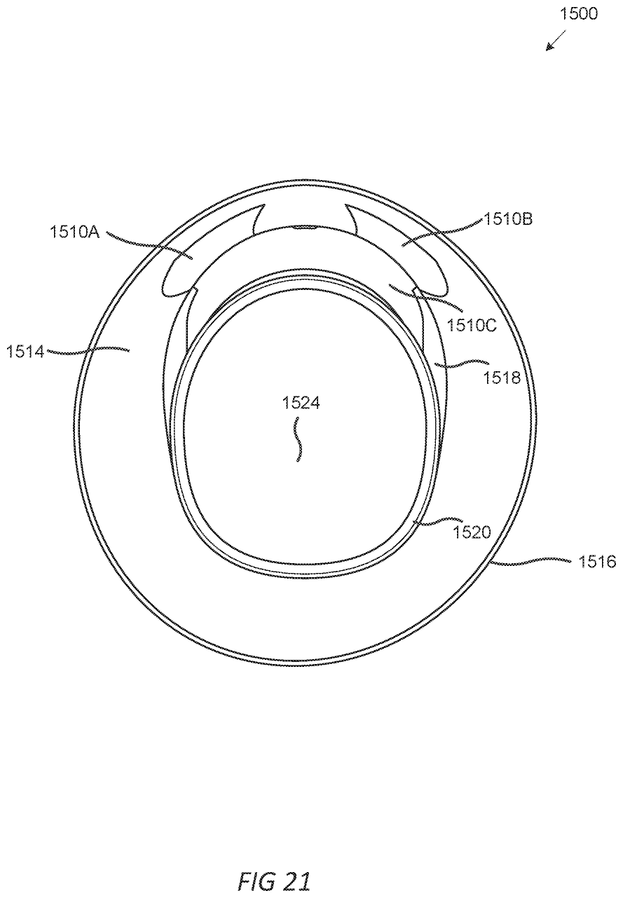

FIG. 21 illustrates a bottom view of the exemplary joint stabilization knee sleeve 1500, according to one embodiment of the present disclosure. As shown in the present embodiment, the lower 1518 and upper 1514 portions of the sleeve include regions 1516 and 1520, where these regions are made of the base materials polyester spandex and/or breathable cotton. In some embodiments, the lower portion 1518 includes an opening 1524 to which the foot of the wearer protrudes. In particular embodiments, the regions 1510A and 1510B are designed to extend downwards from the upper thigh of the wearer towards region 1510C of the knee for providing support and compression benefits similar to those achieved via medical taping practices that include wrapping tape around the knee and thigh.

### Alternative Aspects

According to a first aspect, the present disclosure discusses embodiments of a technical garment, the technical garment including: a garment body including a first end and a second end and forming at least one opening for receiving a particular body region; a base material manufactured from one or more fiber types selected from the group including cotton, elastic, lycra, and spandex, wherein the base material is expandable to at least a size of the particular body region; and one or more compressive regions including one or more technical fibers interwoven with the base material in one or more patterns, wherein the one or more technical fibers include a tensile strength greater than a tensile strength of the one or more fiber types of the base material for providing compression via resisting expansion in response to a user inserting the particular body region into the at least one opening, and wherein the one or more patterns include two or more targeted compressive regions extending along a length of the technical garment for at least partially preventing movement of the particular body region when covered by the one or more compressive regions.

According to a second aspect, the technical garment of the first aspect or any other aspect, wherein a portion of the particular body region covered by the one or more compressive regions is located medially to the two or more targeted compressive regions.

According to a third aspect, the technical garment of the second aspect or any other aspect, wherein the two or more targeted compressive regions apply semi-opposing compression at least laterally to the portion of the particular body region.

According to a fourth aspect, the technical garment of the first aspect or any other aspect, wherein the at least one opening is a first opening of at least two openings.

According to a fifth aspect, the technical garment of the fourth aspect or any other aspect, wherein the first opening includes a largest opening diameter of the at least two openings.

According to a sixth aspect, the technical garment of the first aspect or any other aspect, wherein a third targeted compression region of the two or more targeted compression regions integrates with the two or more targeted compression regions at at least one end of the two or more targeted compression regions.

According to a seventh aspect, the technical garment of the first aspect or any other aspect, wherein the two or more targeted compressive regions apply a pressure to the portion of the particular body region when worn, wherein the pressure is approximately 10-100 mmHG.

According to an eighth aspect, the technical garment of the seventh aspect or any other aspect, wherein the technical garment is a sock and the two or more targeted compressive regions apply a pressure of approximately 23-93 mmHg to the portion of the particular body region.

According to a ninth aspect, the technical garment of the seventh aspect or any other aspect, wherein the technical garment is an elbow sleeve and the two or more targeted compressive regions apply a pressure of approximately 23-29 mmHg to the portion of the particular body region.

According to a tenth aspect, the technical garment of the seventh aspect or any other aspect, wherein the technical garment is a knee sleeve and the two or more targeted compressive regions apply a pressure of approximately 23-93 mmHg to the portion of the particular body region.

According to an eleventh aspect, the technical garment of the seventh aspect or any other aspect, wherein the technical garment is a pair of shorts and the two or more targeted compressive regions apply a pressure of approximately 74-93 mmHg to the portion of the particular body region.

According to a twelfth aspect, the technical garment of the seventh aspect or any other aspect, wherein the technical garment is a shirt and the two or more targeted compressive regions apply a pressure of approximately 11-30 mmHg to the portion of the particular body region.

According to a thirteenth aspect, the technical garment of the first aspect or any other aspect, wherein the one or more patterns include the one or more technical fibers woven as a closed basket-weave.

According to a fourteenth aspect, the technical garment of the first aspect or any other aspect, wherein the one or more patterns include the one or more technical fibers woven in a substantially-wishboned shape.

According to a fifteenth aspect, the technical garment of the first aspect or any other aspect, wherein the one or more patterns include the one or more technical fibers woven as a plurality of intersecting arches.

According to a sixteenth aspect, the technical garment of the first aspect or any other aspect, wherein the garment body further includes a cuff extending along an outer circumference of the at least one opening.

According to a seventeenth aspect, the technical garment of the sixteenth aspect or any other aspect, wherein the cuff includes a semi-adhesive inner surface for maintaining a position of the cuff on a wearer's skin at the particular body region.

According to an eighteenth aspect, the technical garment of the seventeenth aspect or any other aspect, wherein the semi-adhesive inner surface includes surface ribs for maintaining the position of the cuff on the wearer's skin at the particular body region.

According to a nineteenth aspect, the technical garment of the first aspect or any other aspect, wherein the garment body includes a substantially-tubular shape.

According to a twentieth aspect, a garment is disclosed, the garment including: a first end and a second end with a generally tubular body therebetween, wherein: the generally tubular body defines at least one opening; the generally tubular body includes a first region and a second region; the first region includes a material of a first tensile strength; the second region includes a material of a second tensile strength; the second tensile strength is greater than the first tensile strength; and the second region forms a pattern including at least two compressive areas, the at least two compressive areas extending along at least a portion of a length of the generally tubular body and at least partially surrounding a portion of the first region.

According to a twenty-first aspect, the garment of the twentieth aspect or any other aspect, wherein the garment is a sock.

According to a twenty-second aspect, the garment of the twenty-first aspect or any other aspect, wherein the first region includes an area for a wearer's toes when the sock is worn.

According to a twenty-third aspect, the garment of the twenty-second aspect or any other aspect, wherein the first region includes an area for the wearer's heal when the sock is worn.

According to a twenty-fourth aspect, the garment of the twenty-third aspect or any other aspect, wherein the first region includes an area for at least a portion of the wearer's shin when the sock is worn.

According to a twenty-fifth aspect, the garment of the twenty-fourth aspect or any other aspect, wherein the at least two compressive areas are positioned to cover at least a portion of the wearer's shin when the sock is worn.

According to a twenty-sixth aspect, the garment of the twenty-fifth aspect or any other aspect, wherein a portion of the second region is positioned to cover at least a portion of the wearer's Achilles tendon when the sock is worn.

According to a twenty-seventh aspect, the garment of the twentieth aspect or any other aspect, wherein the generally tubular body defines two openings.

According to a twenty-eighth, the garment of the twenty-seventh aspect or any other aspect, wherein the garment is a knee sleeve.

According to a twenty-ninth aspect, the garment of the twenty-eighth aspect or any other aspect, wherein the first region includes an area for at least a portion of a wearer's knee when the knee sleeve is worn.

According to a thirtieth aspect, the garment of the twenty-ninth aspect or any other aspect, wherein the at least two compressive areas are positioned to cover at least a portion of the wearer's knee when the knee sleeve is worn.

According to a thirty-first aspect, the garment of the thirtieth aspect or any other aspect, wherein the at least two compressive areas are positioned proximate the wearer's knee cap when the knee sleeve is worn.

According to a thirty-second aspect, the garment of the thirty-first aspect or any other aspect, wherein the second region includes a third area positioned near a bottom of the wearer's knee cap when the knee sleeve is worn.

According to a thirty-third aspect, the garment of the twenty-seventh aspect or any other aspect, wherein the garment is an elbow sleeve.

According to a thirty-fourth aspect, the garment the thirty-third aspect or any other aspect, wherein the at least two compressive areas are positioned proximate a wearer's elbow when the elbow sleeve is worn.

According to a thirty-fifth aspect, the garment of the thirty-fourth aspect or any other aspect, wherein the at least two compressive areas surround at least a portion of the wearer's elbow when the elbow sleeve is worn.

According to a thirty-sixth aspect, the garment of the thirty-fifth aspect or any other aspect, wherein the at least two compressive areas form a substantially diamond-shaped pattern.

According to a thirty-seventh aspect, the garment of the thirty-fifth aspect or any other aspect, wherein the at least two compressive areas form a semi-circular pattern.

According to a thirty-eighth aspect, the garment of the twentieth aspect or any other aspect, wherein the generally tubular body includes at least one cuffed-region around the at least one opening.

According to a thirty-ninth aspect, the garment of the twentieth aspect or any other aspect, wherein the generally tubular body includes an interior portion for receiving a portion of a wearer's body.

According to a fortieth aspect, a sock is disclosed including: a first end and a second end with a generally tubular body therebetween, wherein: the generally tubular body defines at least one opening; the generally tubular body includes a first region and a second region; the first region includes a material of a first tensile strength; the second region includes a material of a second tensile strength; the second tensile strength is greater than the first tensile strength; and the second region forms a pattern including at least two compressive areas, the at least two compressive areas extending along at least a portion of the length of the generally tubular body and at least partially surrounding a portion of the first region.

According to a forty-first aspect, the sock of the fortieth aspect or any other aspect, wherein the first region includes an area for a wearer's toes when the sock is worn.

According to a forty-second aspect, the sock of the forty-first aspect or any other aspect, wherein the first region includes an area for the wearer's heal when the sock is worn.

According to a forty-third aspect, the sock of the forty-second aspect or any other aspect, wherein the first region includes an area for at least a portion of the wearer's shin when the sock is worn.

According to a forty-fourth aspect, the sock of the forty-third aspect or any other aspect, wherein the at least two compressive areas are positioned to cover at least a portion of the wearer's shin when the sock is worn.

According to a forty-fifth aspect, the sock of the forty-fourth aspect or any other aspect, wherein a portion of the second region is positioned to cover at least a portion of the wearer's Achilles tendon when the sock is worn.

According to a forty-sixth aspect, an arm sleeve is disclosed including: a first end and a second end with a generally tubular body therebetween, wherein: the generally tubular body defines two openings; the generally tubular body includes a first region and a second region; the first region includes a material of a first tensile strength; the second region includes a material of a second tensile strength; the second tensile strength is greater than the first tensile strength; and the second region forms a pattern including at least two compressive areas, the at least two compressive areas extending along at least a portion of the length of the generally tubular body and at least partially surrounding a portion of the first region.

According to a forty-seventh aspect, the arm sleeve of the forty-sixth aspect or any other aspect, wherein the at least two compressive areas are positioned proximate a wearer's elbow when the elbow sleeve is worn.

According to a forty-eighth aspect, the arm sleeve of the forty-seventh aspect or any other aspect, wherein the at least two compressive areas surround at least a portion of the wearer's elbow when the elbow sleeve is worn.

According to a forty-ninth aspect, the arm sleeve of the forty-eighth aspect or any other aspect, wherein the at least two compressive areas form a substantially diamond-shaped pattern.

According to a fiftieth aspect, the arm sleeve of the forty-eighth aspect or any other aspect, wherein the at least two compressive areas form a semi-circular pattern.

According to a fifty-first aspect, a knee sleeve is disclosed including: a first end and a second end with a generally tubular body therebetween, wherein: the generally tubular body defines two openings; the generally tubular body includes a first region and a second region; the first region includes a material of a first tensile strength; the second region includes a material of a second tensile strength; the second tensile strength is greater than the first tensile strength; and the second region forms a pattern including at least two compressive areas, the at least two compressive areas extending along at least a portion of the length of the generally tubular body and at least partially surrounding a portion of the first region.

According to a fifty-second aspect, the knee sleeve of the fifty-first aspect or any other aspect, wherein the first region includes an area for at least a portion of a wearer's knee when the knee sleeve is worn.

According to a fifty-third aspect, the knee sleeve of the fifty-second aspect or any other aspect, wherein the at least two compressive areas are positioned to cover at least a portion of the wearer's knee when the knee sleeve is worn.

According to a fifty-fourth aspect, the knee sleeve of the fifty-third aspect or any other aspect, wherein the at least two compressive areas are positioned proximate the wearer's knee cap when the knee sleeve is worn.

According to a fifty-fifth aspect, the knee sleeve of the fifty-fourth aspect or any other aspect, wherein the second region includes a third area positioned near a bottom of the wearer's knee cap when the knee sleeve is worn.

### Conclusion

While various aspects have been described in the context of a preferred embodiment, additional aspects, features, and methodologies of the claimed embodiments will be readily discernible from the description herein, by those of ordinary skill in the art. Many embodiments and adaptations of the disclosure and claimed embodiments other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed embodiments. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed embodiments. In addition, some steps may be carried out simultaneously, contemporaneously, or in synchronization with other steps.

The embodiments were chosen and described in order to explain various principles and practical applications of the embodiments so as to enable others skilled in the art to utilize the various embodiments and with various modifications as are suited to the particular use contemplated. Alternative embodiments will become apparent to those skilled in the art to which the claimed embodiments pertain without departing from their spirit and scope. Accordingly, the scope of the claimed embodiment is defined by the appended claims rather than the foregoing description and the exemplary embodiments described therein.

6. The technical garment of claim 1 or any other claim, wherein a third targeted compression region of the two or more targeted compression regions integrates with the two or more targeted compression regions at at least one end of the two or more targeted compression regions.

7. The technical garment of claim 3 or any other claim, wherein the two or more targeted compressive regions apply a pressure to the portion of the particular body region when worn, wherein the pressure is approximately 10-100 mmHg.

8. The technical garment of claim 7 or any other claim, wherein the technical garment is a sock and the two or more targeted compressive regions apply a pressure of approximately 23-93 mmHg to the portion of the particular body region.

9. The technical garment of claim 7 or any other claim, wherein the technical garment is an elbow sleeve and the two or more targeted compressive regions apply a pressure of approximately 23-29 mmHg to the portion of the particular body region.

10. The technical garment of claim 7 or any other claim, wherein the technical garment is a knee sleeve and the two or more targeted compressive regions apply a pressure of approximately 23-93 mmHg to the portion of the particular body region.

11. The technical garment of claim 7 or any other claim, wherein the technical garment is a pair of shorts and the two or more targeted compressive regions apply a pressure of approximately 74-93 mmHg to the portion of the particular body region.

12. The technical garment of claim 7 or any other claim, wherein the technical garment is a shirt and the two or more targeted compressive regions apply a pressure of approximately 11-30 mmHg to the portion of the particular body region.

13. The technical garment of claim 1 or any other claim, wherein the one or more patterns comprise the one or more technical fibers woven as a closed basket-weave.

14. The technical garment of claim 1 or any other claim, wherein the one or more patterns comprise the one or more technical fibers woven in a substantially-wishboned shape.

15. The technical garment of claim 1 or any other claim, wherein the one or more patterns comprise the one or more technical fibers woven as a plurality of intersecting arches.

16. The technical garment of claim 1 or any other claim, wherein the garment body further comprises a cuff extending along an outer circumference of the at least one opening.

17. The technical garment of claim 16 or any other claim, wherein the cuff comprises a semi-adhesive inner surface for maintaining a position of the cuff on a wearer's skin at the particular body region.

18. The technical garment of claim 17 or any other claim, wherein the semi-adhesive inner surface comprises surface ribs for maintaining the position of the cuff on the wearer's skin at the particular body region.

19. The technical garment of claim 1 or any other claim, wherein the garment body comprises a substantially-tubular shape.

20. A garment comprising:
a first end and a second end with a generally tubular body therebetween, wherein:
the generally tubular body defines at least one opening;
the generally tubular body comprises a first region and a second region;
the first region comprises a material of a first tensile strength;
the second region comprises a material of a second tensile strength;
the second tensile strength is greater than the first tensile strength; and
the second region forms a pattern comprising at least two compressive areas, the at least two compressive areas extending along at least a portion of a length of the generally tubular body and at least partially surrounding a portion of the first region.

21. The garment of claim 20 or any other claim, wherein the garment is a sock.

22. The garment of claim 21 or any other claim, wherein the first region comprises an area for a wearer's toes when the sock is worn.

23. The garment of claim 22 or any other claim, wherein the first region comprises an area for the wearer's heal when the sock is worn.

24. The garment of claim 23 or any other claim, wherein the first region comprises an area for at least a portion of the wearer's shin when the sock is worn.

25. The garment of claim 24 or any other claim, wherein the at least two compressive areas are positioned to cover at least a portion of the wearer's shin when the sock is worn.

26. The garment of claim 25 or any other claim, wherein a portion of the second region is positioned to cover at least a portion of the wearer's Achilles tendon when the sock is worn.

27. The garment of claim 20 or any other claim, wherein the generally tubular body defines two openings.

28. The garment of claim 27 or any other claim, wherein the garment is a knee sleeve.

29. The garment of claim 28 or any other claim, wherein the first region comprises an area for at least a portion of a wearer's knee when the knee sleeve is worn.

30. The garment of claim 29 or any other claim, wherein the at least two compressive areas are positioned to cover at least a portion of the wearer's knee when the knee sleeve is worn.

31. The garment of claim 30 or any other claim, wherein the at least two compressive areas are positioned proximate the wearer's knee cap when the knee sleeve is worn.

32. The garment of claim 31 or any other claim, wherein the second region comprises a third area positioned near a bottom of the wearer's knee cap when the knee sleeve is worn.

33. The garment of claim 27 or any other claim, wherein the garment is an elbow sleeve.

34. The garment of claim 33 or any other claim, wherein the at least two compressive areas are positioned proximate a wearer's elbow when the elbow sleeve is worn.

35. The garment of claim 34 or any other claim, wherein the at least two compressive areas surround at least a portion of the wearer's elbow when the elbow sleeve is worn.

36. The garment of claim 35 or any other claim, wherein the at least two compressive areas form a substantially diamond-shaped pattern.

37. The garment of claim 35 or any other claim, wherein the at least two compressive areas form a semi-circular pattern.

38. The garment of claim 20 or any other claim, wherein the generally tubular body comprises at least one cuffed-region around the at least one opening.

39. The garment of claim 20 or any other claim, wherein the generally tubular body comprises an interior portion for receiving a portion of a wearer's body.

40. A sock comprising:
a first end and a second end with a generally tubular body therebetween, wherein:
the generally tubular body defines at least one opening;
the generally tubular body comprises a first region and a second region;
the first region comprises a material of a first tensile strength;
the second region comprises a material of a second tensile strength;
the second tensile strength is greater than the first tensile strength; and
the second region forms a pattern comprising at least two compressive areas, the at least two compressive areas extending along at least a portion of a length of the generally tubular body and at least partially surrounding a portion of the first region.

41. The sock of claim 40 or any other claim, wherein the first region comprises an area for a wearer's toes when the sock is worn.

42. The sock of claim 41 or any other claim, wherein the first region comprises an area for the wearer's heal when the sock is worn.

43. The sock of claim 42 or any other claim, wherein the first region comprises an area for at least a portion of the wearer's shin when the sock is worn.

44. The sock of claim 43 or any other claim, wherein the at least two compressive areas are positioned to cover at least a portion of the wearer's shin when the sock is worn.

45. The sock of claim 44 or any other claim, wherein a portion of the second region is positioned to cover at least a portion of the wearer's Achilles tendon when the sock is worn.

46. An arm sleeve comprising:
a first end and a second end with a generally tubular body therebetween, wherein:
the generally tubular body defines two openings;
the generally tubular body comprises a first region and a second region;
the first region comprises a material of a first tensile strength;
the second region comprises a material of a second tensile strength;
the second tensile strength is greater than the first tensile strength; and
the second region forms a pattern comprising at least two compressive areas, the at least two compressive areas extending along at least a portion of a length of the generally tubular body and at least partially surrounding a portion of the first region.

47. The arm sleeve of claim 46 or any other claim, wherein the at least two compressive areas are positioned proximate a wearer's elbow when the elbow sleeve is worn.

48. The arm sleeve of claim 47 or any other claim, wherein the at least two compressive areas surround at least a portion of the wearer's elbow when the elbow sleeve is worn.

49. The arm sleeve of claim 48 or any other claim, wherein the at least two compressive areas form a substantially diamond-shaped pattern.

50. The arm sleeve of claim 48 or any other claim, wherein the at least two compressive areas form a semi-circular pattern.

51. A knee sleeve comprising:
a first end and a second end with a generally tubular body therebetween, wherein:
the generally tubular body defines two openings;
the generally tubular body comprises a first region and a second region;
the first region comprises a material of a first tensile strength;
the second region comprises a material of a second tensile strength;
the second tensile strength is greater than the first tensile strength; and
the second region forms a pattern comprising at least two compressive areas, the at least two compressive areas extending along at least a portion of a length of the generally tubular body and at least partially surrounding a portion of the first region.

52. The knee sleeve of claim 51 or any other claim, wherein the first region comprises an area for at least a portion of a wearer's knee when the knee sleeve is worn.

53. The knee sleeve of claim 52 or any other claim, wherein the at least two compressive areas are positioned to cover at least a portion of the wearer's knee when the knee sleeve is worn.

54. The knee sleeve of claim 53 or any other claim, wherein the at least two compressive areas are positioned proximate the wearer's knee cap when the knee sleeve is worn.

55. The knee sleeve of claim 54 or any other claim, wherein the second region comprises a third area positioned near a bottom of the wearer's knee cap when the knee sleeve is worn.
